SEQUENCE LISTING (6 sequences)

SEQ ID NO: 1 - DNA, 23 bp, Artificial sequence
  Synthetic primer
cattgctgacaggatgcagaagg

SEQ ID NO: 2 - DNA, 22 bp, Artificial sequence
  Synthetic primer
tgctggaaggtggacagtgagg

SEQ ID NO: 3 - DNA, 24 bp, Artificial sequence
  Synthetic primer
cctagtgataaggaatgcacgatg

SEQ ID NO: 4 - DNA, 23 bp, Artificial sequence
  Synthetic primer
ctaggcaggtttgatctccgttc

SEQ ID NO: 5 - DNA, 22 bp, Artificial sequence
  Synthetic primer
atcatccctgcgagcctatcct

SEQ ID NO: 6 - DNA, 24 bp, Artificial sequence
  Synthetic primer
gaccttttttggctaaacgctttc

(19) United States
(12) Patent Application Publication
(10) Pub. No.: US 2021/0260051 A1
(43) Pub. Date: Aug. 26, 2021

(54) COMBINATION OF EZH2 INHIBITOR AND CHECKPOINT THERAPY FOR THE TREATMENT OF CANCER

(71) Applicant: Board of Regents, The University of Texas System, Austin, TX (US)

(72) Inventors: Padmanee SHARMA, Houston, TX (US); James P. ALLISON, Houston, TX (US); Sangeeta GOSWAMI, Houston, TX (US)

(73) Assignee: Board of Regents, The University of Texas System, Austin, TX (US)

(21) Appl. No.: 17/252,002

(22) PCT Filed: Jun. 14, 2019
(86) PCT No.: PCT/US2019/037367

§ 371 Date: Dec. 14, 2020

Related U.S. Application Data
(60) Provisional application No. 62/685,239, filed on Jun. 14, 2018.

(51) Int. Cl. A61K 31/4545; A61P 35/00; C07K 16/28; A61K 45/06

(52) U.S. Cl. CPC A61K 31/4545; A61P 35/00; A61K 2039/54; C07K 16/2827; A61K 45/06; C07K 16/2818

(57) ABSTRACT

Provided herein are methods of treating cancer comprising administering an EZH2 inhibitor which may be combined with an immune checkpoint inhibitor. Further provided herein are methods of depleting regulatory T cells (Tregs) in a subject comprising administering an EZH2 inhibitor to the subject. Also provided herein are pharmaceutical compositions comprising CPI-1205 and an immune checkpoint inhibitor.

This application claims the benefit of U.S. Provisional Patent Application No. 62/685,239, filed Jun. 14, 2018, the entire contents of which are incorporated herein by reference.

The sequence listing that is contained in the file named “UTFCP1337WO.txt”, which is 2 KB (as measured in Microsoft Windows) and was created on Jun. 13, 2019, is filed herewith by electronic submission and is incorporated by reference herein.

### BACKGROUND

### 1. Field

The present invention relates generally to the fields of medicine and immunology. More particularly, it concerns the combination therapy of EZH2 inhibitor with immune checkpoint therapy.

### 2. Description of Related Art

Enhancer of zeste homolog 2 (EZH2) is expressed in T cells following its activation via CD28 signaling (DuPage et al., 2015). EZH2 is a catalytic subunit of polycomb repressive complex 2 (PRC2) that trimethylates lysine 27 on histone H3 (H3K27me3), leading to gene repression (Cao et al., 2002; Margueron and Reinberg, 2011). EZH2 can form a complex with FoxP3 that is necessary for maintaining the identity of naturally occurring regulatory T cells following its activation (Arvey et al., 2014; Yang et al., 2015). Further, EZH2 can inhibit T cell differentiation into effector T cells, mostly by conferring repressive marks in key cytokine and transcription factor genes (Kwon et al., 2017; Tumes et al., 2013; Wei et al., 2009; Zhang et al., 2014; Kanno et al., 2012). Overall, EZH2 maintains suppressive activity in T cells that prevents the development of autoimmunity (Coit et al., 2016; Sarmento et al., 2017) but may have a negative impact on anti-tumor immunity. EZH2 has been shown to regulate chemokine expression in cancer cells and immunogenicity of melanoma tumor cells; thereby, EZH2 inhibition can increase T cell infiltration (Nagarsheth et al., 2015; Peng et al., 2015; Zingg et al., 2017). However, the direct role of EZH2-mediated reprograming of T cells in anti-tumor immunity, especially in the context of immune checkpoint therapy, has not been studied.

### SUMMARY

In a first embodiment, the present disclosure provides methods for treating cancer in a subject comprising administering an effective amount of CPI-1205 in combination with an immune checkpoint inhibitor to the subject. In some aspects, the subject is resistant to an immune checkpoint inhibitor. In specific aspects, the subject is human. In some aspects, more than one immune checkpoint inhibitor is administered. In some aspects, the cancer is bladder cancer, melanoma or prostate cancer.

In some embodiments, CPI-1205 is administered to a subject with cancer or being treated for cancer to inhibit an increase in Enhancer of zeste homolog 2 (EZH2) activity mediated by administration of immune checkpoint inhibitor therapy to the subject. In other embodiments, CPI-1205 is administered to a subject with cancer or being treated for cancer to inhibit an immune checkpoint inhibitor therapy mediated increase in Enhancer of zeste homolog 2 (EZH2) activity. In specific embodiments, the immune checkpoint inhibitor therapy mediated increase in EZH2 activity is in T cells, particularly regulatory or suppressive T cells.

In some aspects, the subject has one or more tumors. In certain aspects, the administration of CPI-1205 and immune checkpoint inhibitor results in reduced tumor growth or a reduction in tumor mass. In particular aspects, the administration of CPI-1205 and immune checkpoint inhibitor results in greater reduction in tumor growth or greater reduction in tumor mass relative to administration of immune checkpoint inhibitor therapy alone.

In certain aspects, the CPI-1205 and/or the immune checkpoint inhibitor are administered orally, intravenously, intraperitoneally, intratracheally, intratumorally, intramuscularly, endoscopically, intralesionally, percutaneously, subcutaneously, regionally, or by direct injection or perfusion. In specific aspects, the CPI-1205 is administered orally and the immune checkpoint inhibitor is administered intravenously. In some aspects, the CPI-1205 and/or the immune checkpoint inhibitor are administered more than once. In certain aspects, the CPI-1205 and/or the immune checkpoint inhibitor are administered daily. In some aspects, the CPI-1205 and the immune checkpoint inhibitor are administered concurrently. In some aspects, the CPI-1205 is administered before the immune checkpoint inhibitor. In other aspects, the CPI-1205 is administered after the immune checkpoint inhibitor.

In some aspects, the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR). In particular aspects, the immune checkpoint inhibitor is a PD-1 inhibitor. In some aspects, the PD-1 inhibitor is nivolumab, pembrolizumab, CT-011, BMS 936559, MPDL328OA or AMP-224. In certain aspects, the immune checkpoint inhibitor is a CTLA-4 inhibitor. In some aspects, the CTLA-4 inhibitor is ipilimumab or tremelimumab.

In specific embodiments, CPI-1205 is administered at an 800 mg twice daily oral dose and ipilimumab is administered intravenously at a dose of 3 mg/kg every 3 weeks. In specific embodiments, an effective dose of CPI-1205 may be an oral twice daily dose of 200 mg, 400 mg 800 mg or 1600 mg provided in 7, 14, 21, or 28 day cycles, or 1, 2, 3, 4, 5, 6, 7, 8, 9, 10, 11 or 12 month cycles. In still other embodiments, an effective dose of CPI-1205 may be a 10 mg, 25 mg, 50 mg, 75 mg, 100 mg, 200 mg, 300 mg, 400 mg, 500 mg, 600 mg, 700 mg, 800 mg, 900 mg, 1000 mg, 1100 mg, 1200 mg 1300 mg 1400 mg, 1500 mg, 1600 mg, 1700 mg 1800 mg, 1900 mg or 2000 mg dose of CPI-1205 administered once, twice, 3, 4, 5 or 6 times a day. Specifically, CPI-1205 may be provided in 7, 14, 21 or 28 day cycles, or 1, 2, 3, 4, 5, 6, 7, 8, 9, 10, 11, 12, 13, 14, 15, 16, 17, 28, 19, 20, 21, 22, 23, 24, 30 or 36 month cycles, or 1, 2, 3, 4 or 5 year cycles.

In some embodiments, the ipilimumab is administered at an effective dose of 3 mg/kg or 10 mg/kg. In specific embodiments, the ipilimumab is administered at an effective dose of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, or 15 mg/kg. Specifically, ipilimumab may be provided intravenously over 90 minutes every 3 weeks to 12 weeks for 1, 2, 3, 4, 5, 6, 7, 8, 9, 10, or more doses. The ipilimumab may be administered every 1 week, 2 weeks, 3 weeks, 4 weeks, 5 weeks, 6, weeks, 7 weeks, 8 weeks, 9 weeks, 10 weeks, 11 weeks, 12 weeks, 13 weeks, 14 weeks or 15 weeks. In some embodiments, ipilimumab is administered at a dose of 10 mg/kg administered intravenously over 90 minutes every 3 weeks for 4 doses followed by 10 mg/kg every 12 weeks for up to 3 years.

In some embodiments, the nivolumab is administered at an effective dose of 240 mg or 3 mg/kg as an intravenous infusion. Specifically, the nivolumab may be administered at a dose of 240 mg as an intravenous infusion over 30 minutes every 2 weeks. In specific embodiments, an effective dose of nivolumab may be 200 mg, 220 mg, 240 mg or 260 mg provided in 7, 14, 21 or 28 day cycles, or 1, 2, 3, 4, 5, 6, 7, 8, 9, 10, 11 or 12 month cycles. In specific embodiments, an effective dose of nivolumab may be 1 mg/kg, 2 mg/kg, 3 mg/kg, 4 mg/kg, or 5 mg/kg. Specifically, the intravenous infusion may be over 10, 20, 30, 40, 50, 60, 70, 80, or 90 minutes. In still other embodiments, an effective dose of nivolumab may be a 10 mg, 25 mg, 50 mg, 75 mg, 100 mg, 200 mg, 300 mg, 400 mg, 500 mg, 600 mg, 700 mg, 800 mg, 900 mg, 1000 mg, 1100 mg, 1200 mg 1300 mg 1400 mg, 1500 mg, 1600 mg, 1700 mg 1800 mg, 1900 mg or 2000 mg dose of nivolumab administered once, twice, 3, 4, 5 or 6 times a day. Specifically, nivolumab may be provided in 7, 14, 21 or 28 day cycles, or 1, 2, 3, 4, 5, 6, 7, 8, 9, 10, 11, 12, 13, 14, 15, 16, 17, 28, 19, 20, 21, 22, 23, 24, 30 or 36 month cycles, or 1, 2, 3, 4 or 5 year cycles.

In some embodiments, the pembrolizumab is administered at an effective dose of 200 mg or 2 mg/kg. Specifically, the pembrolizumab may be administered at an effective dose of 200 mg as an invtravenous infusion every 30 minutes every 3 weeks. In specific embodiments, an effective dose of pembrolizumab may be a dose of 100 mg, 200 mg, 300 mg, 400 mg or 500 mg provided in 7, 14, 21 or 28 day cycles, or 1, 2, 3, 4, 5, 6, 7, 8, 9, 10, 11 or 12 month cycles. In specific embodiments, an effective dose of pembrolizumab may be 1 mg/kg, 2 mg/kg, 3 mg/kg, 4 mg/kg, or 5 mg/kg. In still other embodiments, an effective dose of pembrolizumab may be a 10 mg, 25 mg, 50 mg, 75 mg, 100 mg, 200 mg, 300 mg, 400 mg, 500 mg, 600 mg, 700 mg, 800 mg, 900 mg, 1000 mg, 1100 mg, 1200 mg 1300 mg 1400 mg, 1500 mg, 1600 mg, 1700 mg 1800 mg, 1900 mg or 2000 mg dose of pembrolizumab administered once, twice, 3, 4, 5 or 6 times a day. Specifically, pembrolizumab may be provided in 7, 14, 21 or 28 day cycles, or 1, 2, 3, 4, 5, 6, 7, 8, 9, 10, 11, 12, 13, 14, 15, 16, 17, 28, 19, 20, 21, 22, 23, 24, 30 or 36 month cycles, or 1, 2, 3, 4 or 5 year cycles.

In some embodiments, the tremelimumab is administered at an effective dose of 3 mg/kg, 6 mg/kg, or 10 mg/kg. In specific embodiments, an effective dose of tremelimumab may be 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg provided in 7, 14, 21 or 28 day cycles, or 1, 2, 3, 4, 5, 6, 7, 8, 9, 10, 11 or 12 month cycles. In still other embodiments, an effective dose of tremelimumab may be a 10 mg, 25 mg, 50 mg, 75 mg, 100 mg, 200 mg, 300 mg, 400 mg, 500 mg, 600 mg, 700 mg, 800 mg, 900 mg, 1000 mg, 1100 mg, 1200 mg 1300 mg 1400 mg, 1500 mg, 1600 mg, 1700 mg 1800 mg, 1900 mg or 2000 mg dose of tremelimumab administered once, twice, 3, 4, 5 or 6 times a day. Specifically, tremelimumab may be provided in 7, 14, 21 or 28 day cycles, or 1, 2, 3, 4, 5, 6, 7, 8, 9, 10, 11, 12, 13, 14, 15, 16, 17, 28, 19, 20, 21, 22, 23, 24, 30 or 36 month cycles, or 1, 2, 3, 4 or 5 year cycles.

In specific embodiments, CPI-1205 is administered orally before ipilimumab is administered intravenously. Specifically, the CPI-1205 may be administered 1, 2, 3, 4, 5, 6, 7, 8, 9, 10, 12, 24, 36, 48, or 72 hours, or 4, 5, 6, 7, 8, 9, 10, 11, 12, 13, or 14 days, or 3, 4, 5, 6, 7, or 8 weeks, or 3, 4, 5, 6, 7, 8, 9, 10, 11, or 12 months, or 1.5, 2, 2.5, 3, 3.5, 4, 4.5 or 5 years prior to ipilimumab. In specific embodiments, CPI-1205 is administered orally after ipilimumab is administered intravenously. Specifically, the CPI-1205 may be administered 1, 2, 3, 4, 5, 6, 7, 8, 9, 10, 12, 24, 36, 48, or 72 hours, or 4, 5, 6, 7, 8, 9, 10, 11, 12, 13, or 14 days, or 3, 4, 5, 6, 7, or 8 weeks, or 3, 4, 5, 6, 7, 8, 9, 10, 11, or 12 months, or 1.5, 2, 2.5, 3, 3.5, 4, 4.5 or 5 years after ipilimumab. In some embodiments, CPI-1205 may be administered concurrently with ipilimumab. Specifically, the first CPI-1205 may be administered within 1, 2, 3, 4, 5, 6, 7, 8, 9, or 10 hours of the first dose of ipilimumab.

In certain aspects, the administration results in an increase in CD8⁺ interferon (IFN)-γ⁺ T cells, CD8⁺ granzyme B (GzB)⁺ T cells and/or CD8⁺ tumor necrosis factor (TNF)-α⁺ T cells. In some aspects, the administration results in an increase of intra-tumoral T cells. In certain aspects, the intra-tumoral T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺ or additional combinations of these markers (e.g., CD8⁺ FoxP3⁺IFNγ⁺, CD8⁺IFN-γ⁺, GzB⁺, CD8⁺IFN-γ⁺ TNF-α⁺). In particular aspects, the administration results in an increased ratio of effector T cells to regulatory T cells intratumorally. In some aspects, the administration results in increased infiltration of T cells into the one or more tumors. In particular aspects, the T cells are effector T cells. In specific aspects, the T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺. In some aspects, the administration results in a decrease in suppressive T cells. In particular aspects, the administration results in a decrease in inducible regulatory T cell differentiation. In certain aspects, the administration results in a decrease in FOXP3, NRP1, and/or BACH2 expression.

In additional aspects, the method further comprises the step of administering at least one additional therapeutic agent to the subject. In some aspects, the subject receives at least one additional type of therapy. In certain aspects, the at least one additional type of therapy is selected from the group consisting of chemotherapy, radiotherapy, and immunotherapy.

In another embodiment, there are provided methods of treating cancer in a subject comprising administering an Enhancer of zeste homolog 2 (EZH2) inhibitor to the subject, wherein the patient has been determined to be resistant to immune checkpoint therapy. In some aspects, the EZH2 inhibitor is CPI-1205.

In additional aspects, the method further comprises administering an immune checkpoint inhibitor. In some aspects, the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR). In particular aspects, the immune checkpoint inhibitor is a PD-1 inhibitor. In some aspects, the PD-1 inhibitor is nivolumab, pembrolizumab, CT-011, BMS 936559, MPDL328OA or AMP-224. In certain aspects, the immune checkpoint inhibitor is a CTLA-4 inhibitor. In some aspects, the CTLA-4 inhibitor is ipilimumab or tremelimumab.

A further embodiment provides methods of depleting regulatory T cells (Tregs) in a subject comprising administering an EZH2 inhibitor to the subject. In some aspects, the Tregs are inducible regulatory T cells. In certain aspects, the Tregs are FoxP3⁺ Tregs. In some aspects, the EZH2 inhibitor is CPI-1205.

In additional aspects, the method further comprises administering an immune checkpoint inhibitor. In some aspects, the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR). In certain aspects, the immune checkpoint inhibitor is a PD-1 inhibitor. In some aspects, the PD-1 inhibitor is nivolumab, pembrolizumab, CT-011, BMS 936559, MPDL328OA or AMP-224. In certain aspects, the immune checkpoint inhibitor is a CTLA-4 inhibitor. In some aspects, the CTLA-4 inhibitor is ipilimumab or tremelimumab.

Another embodiment provides a pharmaceutical composition comprising CPI-1205 and an immune checkpoint inhibitor. Further provided herein is the pharmaceutical composition comprising CPI-1205 and an immune checkpoint inhibitor for use in the treatment of cancer. A further embodiment provides the use of a therapeutically effective amount of the CPI-1205 and an immune checkpoint inhibitor for the treatment of cancer. In yet another embodiment there is provided a composition comprising a therapeutically effective amount of CPI-1205 and an immune checkpoint inhibitor for the treatment of cancer in a subject.

Some embodiments provide for the use of an Enhancer of zeste homolog 2 (EZH2) inhibitor and an immune checkpoint inhibitor in the manufacture of a medicament for the treatment of cancer. In specific embodiments, the EZH2 inhibitor is CPI-1205. In still other embodiments, the immune checkpoint inhibitor is ipilimumab. In certain aspects, the cancer is bladder cancer, melanoma or prostate cancer. In yet another embodiment, there is provided a method of treating cancer in a subject comprising inhibiting Enhancer of zeste homolog 2 (EZH2) function in the subject. In some aspects, the subject has one or more tumors. In certain aspects, EZH2 function is inhibited in regulatory T cells. In particular aspects, the regulatory T cells are inducible regulatory T cells. In some aspects, EZH2 function is inhibited by administration of an effective amount of an EZH2 inhibitor. In particular aspects, the EZH2 inhibitor is CPI-1205. In some aspects, the subject is administered an immune checkpoint inhibitor. In some aspects, the cancer is bladder cancer, prostate cancer or melanoma.

In certain aspects, EZH2 inhibition results in a decrease of one or more regulatory T cell specific factors. In some aspects, the one or more regulatory T cell specific factors is FoxP3, BACH2 or neuropilin 1. In certain aspects, EZH2 inhibition results in an increase of one or more effector cytokines or chemokines selected from the group consisting of IFN-gamma, IL-25, Il-17A, IL-10, IL-18, IL-27, GM-CSF, IL-9 and IL-7. In some aspects, the effector cytokines or chemokines are expressed or released by T cells. In still other aspects, EZH2 inhibition results in a decrease in expression in T cells of one or more genes selected from the group consisting of Ilia, Cd70, Tnf, Bach2, Lif, Tnsf11, Il16, Tgfb1, Nrp1, Foxp3, Il9, Tnfsf9 and Tnfsf18. In particular aspects, EZH2 inhibition results in an increase in expression in T cells of one or more genes selected from the group consisting of Il4, Tnfsf13b, Il5, Il3, Tnfsf12, Il21, Tnfsf10, Il13, Il2, Ccr1, Il24, Csf2, Cxcl10, Prbm1, Tgfb3, Il10, Il33, Cxcr6, Ifng, GzB, Il11, Il18, Cdkn2a, ccr2, Tgfb2, Il1b, Il16 and Il15. In specific embodiments, the T cells with decreased or increased gene expression are regulatory T cells. In particular aspects, EZH2 inhibition attenuates inducible regulatory T cell suppressive activity. In certain aspects, the inhibition of EZH2 function and administration of the immune checkpoint inhibitor results in an increase in effector T cells in the tumor.

A further embodiment provides methods of potentiating immune checkpoint inhibitor therapy in a subject comprising administering an effective amount of an Enhancer of zeste homolog 2 (EZH2) inhibitor. In other embodiments, the effectiveness of immune checkpoint inhibitor therapy is enhanced by administering an effective amount of an EZH2 inhibitor to a subject. In still other embodiments, the effectiveness of immune checkpoint inhibitor therapy is increased, raised or amplified by administering an effective amount of an EZH2 inhibitor to a subject. In some aspects, the subject has been administered, is concurrently being administered or will be administered an immune checkpoint inhibitor. In certain aspects, the subject has one or more tumors. In still other aspects, an EZH2 inhibitor is administered to a subject with cancer or being treated for cancer to inhibit an increase in Enhancer of zeste homolog 2 (EZH2) activity mediated by administration of immune checkpoint inhibitor therapy to the subject. In additional aspects, an EZH2 inhibitor is administered to a subject with cancer or being treated for cancer to inhibit an immune checkpoint inhibitor therapy mediated increase in Enhancer of zeste homolog 2 (EZH2) activity. In specific embodiments, the immune checkpoint inhibitor therapy mediated increase in EZH2 activity is in T cells, particularly regulatory or suppressive T cells. In some aspects, the EZH2 inhibitor is CPI-1205. In some aspects, the subject is human. In some aspects, more than one immune checkpoint inhibitor is administered.

In certain aspects, the administration of CPI-1205 and immune checkpoint inhibitor results in reduced tumor growth or a reduction in tumor mass. In some aspects, the administration of CPI-1205 and immune checkpoint inhibitor results in greater reduction in tumor growth or greater reduction in tumor mass relative to administration of immune checkpoint inhibitor therapy alone.

In some aspects, the CPI-1205 is administered orally and the immune checkpoint inhibitor is administered intravenously. In certain aspects, the CPI-1205 and/or the immune checkpoint inhibitor are administered more than once. In specific aspects, the CPI-1205 and/or the immune checkpoint inhibitor are administered daily. In certain aspects, the CPI-1205 and the immune checkpoint inhibitor are administered concurrently. In some aspects, the CPI-1205 is administered before the immune checkpoint inhibitor. In certain aspects, the CPI-1205 is administered after the immune checkpoint inhibitor.

In some aspects, the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR). In certain aspects, the immune checkpoint inhibitor is a PD-1 inhibitor. In some aspects, the PD-1 inhibitor is nivolumab, pembrolizumab, CT-011, BMS 936559, MPDL328OA or AMP-224. In certain aspects, the immune checkpoint inhibitor is a CTLA-4 inhibitor. In particular aspects, the CTLA-4 inhibitor is ipilimumab or tremelimumab.

In some aspects, the administration of CPI-1205 and immune checkpoint inhibitor results in an increase in CD8⁺ interferon (IFN)-γ⁺ T cells, CD8⁺ granzyme B (GzB)⁺ T cells and/or CD8⁺ tumor necrosis factor (TNF)-α⁺ T cells. In certain aspects, the administration of CPI-1205 and immune checkpoint inhibitor results in an increase of intra-tumoral T cells. In certain aspects, the intra-tumoral T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺. In some aspects, the administration of CPI-1205 and immune checkpoint inhibitor results in an increased ratio of effector T cells to regulatory T cells intratumorally.

In some aspects, the administration of CPI-1205 and immune checkpoint inhibitor results in increased infiltration of T cells into the one or more tumors. In some aspects, the T cells are effector T cells. In some aspects, the T cells are CD8⁺ interferon (IFN)-γ⁺, CD8⁺ granzyme B (GzB)⁺, CD8⁺ tumor necrosis factor (TNF)-α⁺ or FoxP3⁺ IFNγ⁺. In particular aspects, the administration of CPI-1205 and immune checkpoint inhibitor results in a decrease in suppressive T cells. In some aspects, the administration of CPI-1205 and immune checkpoint inhibitor results in a decrease in inducible regulatory T cell differentiation. In particular aspects, the administration of CPI-1205 and immune checkpoint inhibitor results in a reduction in the percentage of CD4⁺FoxP3⁺ regulatory T cells intratumorally. In certain aspects, the administration of CPI-1205 and immune checkpoint inhibitor results in an increase in the percentage of intra-tumoral CD4⁺ICOS⁺T-bet+ effector T cells. In certain aspects, the administration of CPI-1205 and immune checkpoint inhibitor results in an increase in the percentage of intra-tumoral CD8⁺IFNγ⁺ effector T cells. In some aspects, the administration of CPI-1205 and immune checkpoint inhibitor results in an increased ratio of effector T cells to regulatory T cells intratumorally. In certain aspects, the administration of CPI-1205 and immune checkpoint inhibitor results in an increased percentage of CD8⁺IFNγ⁺ T cells, CD8⁺TNFα⁺ T cells or both, in lymph nodes.

Additional aspects contemplate a method of identifying a cancer patient as a candidate for treatment with an Enhancer of zeste homolog 2 (EZH2) inhibitor comprising testing a sample from the patient to determine if the patient has elevated EZH2 activity, wherein if the sample exhibits elevated EZH2 activity the patient is a candidate for EZH2 inhibitor therapy. In some aspects, the patient is receiving immune checkpoint inhibitor therapy. In other aspects, the patient will receive immune checkpoint inhibitor therapy. In particular aspects, the elevated EZH2 activity is a result of the immune checkpoint inhibitor therapy. Particularly, the EZH2 inhibitor can be CPI-1205. In other embodiments, the immune checkpoint inhibitor therapy is ipilimumab. In some instances, the elevated EZH2 activity is determined in T cells. In other instances, the sample is a blood sample. In particular aspects, the sample is a T cell sample. In certain embodiments, the elevated EZH2 activity is determined by measuring a decrease of one or more effector cytokines or chemokines selected from the group consisting of IFN-gamma, IL-25, Il-17A, IL-10, IL-18, IL-27, GM-CSF, IL-9 and IL-7 in the sample. In other embodiments, the elevated EZH2 activity is determined by measuring an increase in expression in T cells of one or more genes selected from the group consisting of Il1a, Cd70, Tnf, Bach2, Lif, Tnsf11, Il16, Tgfb 1, Nrp1, Foxp3, Il9, Tnfsf9 and Tnfsf18. In still other embodiments, the elevated EZH2 activity is determined by measuring a decrease in expression in T cells of one or more genes selected from the group consisting of Il4, Tnfsf13b, Il5, Il3, Tnfsf12, Il21, Tnfsf10, Il13, Il2, Ccr1, Il24, Csf2, Cxcl10, Prbm1, Tgfb3, Il10, Il33, Cxcr6, Ifng, GzB, Il11, Il18, Cdkn2a, ccr2, Tgfb2, Il1b, Il16 and 1115. Particularly, if the patient is determined to be a candidate for EZH2 therapy the patient is administered an oral twice daily dose of 800 mg of CPI-1205. In other aspects, the elevated EZH2 activity is determined relative to EZH2 activity in a patient sample collected prior to the patient receiving immune checkpoint inhibitor therapy.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F: EZH2 inhibition by CPI-1205 enhances T cell-mediated anti-tumor immunity. Tumor growth (FIG. 1A) and percentages of intra-tumoral CD8⁺IFNγ⁺ cells, CD8⁺ GzB⁺ cells, and CD8⁺TNFα⁺ (FIG. 1B) and FoxP3⁺IFNγ⁺ cells (FIG. 1C) in MB49 tumor-bearing Ezh2^{fl/+} (n=10) and FoxP3^{Cre}Ezh2^{fl/fl} (n=10) mice.

FIGS. 2A-2C: Ipilimumab increases EZH2 expression in T cells. (FIG. 2A) Unsupervised hierarchical cluster analysis of RNA sequencing data from the peripheral CD4 T cells of patients with metastatic melanoma before and after 3 doses of ipilimumab therapy (week 7). (FIG. 2B) Matched pair analysis of EZH2 expression before and after ipilimumab therapy. (FIG. 2C) Ezh2 expression in peripheral CD4 T cells from patients with metastatic prostate cancer at baseline and after 3 doses of ipilimumab therapy. (2 tailed student t test was used to determine significance; **p<0.01).

FIGS. 3A-3F: Blocking expression of EZH2 mediated by anti-CTLA-4 using CPI-1205 increases the effectiveness of anti-CTLA-4 therapy. Tumor growth (FIG. 3A), survival (FIG. 3B) and percentages of intra-tumoral CD4⁺CD25⁺FoxP3⁺ regulatory T cells, CD4⁺ICOS⁺T-bet⁺ and CD8⁺IFNγ⁺ cells (FIG. 3C) in MB49 tumor-bearing mice treated with vehicle, anti CTLA-4, CPI-1205, or the combination. Tumor growth (FIG. 3D) and percentages of CD4⁺IFNγ⁺ cells and CD8⁺TNFα⁺ cells in the lymph nodes (LN) (FIG. 3E) of MB49 tumor-bearing Ezh2^{fl/+} and FoxP3^{Cre}Ezh2^{fl/fl} mice treated with anti-CTLA-4 on days 7 and 9. (FIG. 3F) Tumor growth and survival of MB49 tumor-bearing Rag-1^{-/-} mice treated with vehicle or CPI-1205. Data are representative of three independent experiments (n=10 in each group; one-way ANOVA was used to determine significance between the groups. **p<0.01).

FIGS. 4A-B: (FIG. 4A) Percentages of inducible regulatory T cells following in vitro differentiation of murine naïve CD4 T cells into regulatory T cells in the presence and absence of various EZH2 inhibitors. (FIG. 4B) Representative counts of viable cells in culture following treatment with EZH2 inhibitors. Data is representative of three independent experiments. One-way ANOVA was used to determine significance between the groups. **p<0.01.

FIGS. 5A-5C: Naïve T cells from FoxP3enhanced green fluorescent protein (eGFP) C57BL/6 mice that were differentiated into iTregs in the presence or absence of CPI-1205. GFP⁺FoxP3⁺ regulatory T cells were subsequently used for RNA sequencing analysis. (FIG. 5A) Ingenuity Pathway Analysis of RNA sequencing data of regulatory T cells differentiated in the presence of DMSO or CPI-1205. (FIG. 5B) Differentially expressed genes (DEGs) seen following differentiation of inducible regulatory T cells in presence of CPI-1205 or DMSO. (FIG. 5C) Luminex-based cytokine analysis of culture supernatant from inducible regulatory T cells following differentiation with CPI-1205 or DMSO. 2 tailed student t test was used to determine significance; NS, not significant. **p<0.01.

FIGS. 6A-6B: EZH2 expression in CD4⁺ cells at baseline (FIG. 6A) and after ipilimumab therapy (FIG. 6B) correlated with time to prostate-specific antigen progression by Spearman correlation (*p<0.05). NS, not significant.

FIG. 7: Flow cytometry analysis of EZH2 expression on CD4⁺CD45RO⁻CD45RA+CCR7⁻ (CD4 T effector, Teff), CD4⁺CD25⁺FoxP3⁺ (T regulatory, T-reg) and CD8 T cells derived from peripheral blood mononuclear cells of patients with metastatic melanoma at baseline and after three doses of ipilimumab. Matched pair analysis of Ezh2 expression before and after ipilimumab therapy. Student t-test was used for significance (**p<0.01, *p<0.05).

FIGS. 8A-8B: Western blot analysis of (FIG. 8A) CD4 and (FIG. 8B) CD8 T cells derived from the spleen and lymph node of CTLA-4^{-/-} mice and wild-type (WT) littermate controls.

FIGS. 9A-9C: (FIG. 9A) Survival curve from representative experiments of B16-F10 tumor-bearing mice treated with vehicle, anti-CTLA-4, CPI-1205, or the combination of anti CTLA-4 and CPI-1205. (FIG. 9B) Absolute numbers of intra-tumoral CD4⁺FoxP3⁺ regulatory T cells and (FIG. 9C) Ratio of CD4 T-effector cells and regulatory T cells from B 16-F10 tumor-bearing mice treated with vehicle, anti-CTLA-4, CPI-1205, or the combination of anti-CTLA-4 and CPI-1205. Data are representative of two independent experiments. n=10 in each group, **p<0.01.

FIGS. 10A-10D: (FIGS. 10A-B) Real-time RT-PCR analysis of CXCL9 and CXCL10 in B16-F10 and MB-49 cell lines following stimulation with vehicle, IFNγ, CPI-1205 or combination of IFNγ and CPI-1205 presented relative to the expression of actin. (FIGS. 10C-D) Relative CXCL9 and CXCL10 expression in tumor of MB49 tumor-bearing mice treated with vehicle, anti-CTLA-4, CPI-1205, or the combination of anti-CTLA-4 and CPI-1205. Data is representative of two independent experiments. (n=5 in each group). Student t-test was used for significance (**p<0.01, *p<0.05). NS, not significant.

### DESCRIPTION OF ILLUSTRATIVE EMBODIMENTS

EZH2-mediated epigenetic regulation of T cell differentiation and regulatory T cell function has been described previously; however, the role of EZH2 in T cell-mediated anti-tumor immunity, especially in the context of immune checkpoint therapy, is not understood. The present studies show that genetic depletion of EZH2 in regulatory T cells (using FoxPreEZH2^{fl/fl} mice) and pharmacological inhibition of EZH2 by CPI-1205 elicited phenotypic and functional alterations of regulatory T cells, leading to an effector-like T cell profile. Further, CPI-1205 enhanced the cytotoxicity of human effector T cells in vitro and the proportion of tumor-infiltrating cytotoxic T cells in vivo in the murine model. It was observed that ipilimumab (a fully human monoclonal immunoglobulin G1 antibody that blocks cytotoxic T lymphocyte associated protein 4 [CTLA-4]) increased EZH2 expression in human T cells across various tumor types and that increased EZH2 expression in T cells inversely correlates with clinical outcome in a cohort of prostate cancer patients. Thus, it was postulated that upregulation of EZH2 mediated by anti CTLA-4 in T cells modulates T cell responses and diminishes the effectiveness of anti-CTLA-4 therapy. It was found that pharmacologic inhibition of EZH2 by CPI-1205 increased effector-like T cell responses and enhanced the effectiveness of anti-CTLA-4 therapy in tumor-bearing mice.

Accordingly, in certain embodiments, the present disclosure provides compositions and methods for the treatment of cancer by a combination treatment of an EZH2 inhibitor and an immune checkpoint therapy.

### I. METHODS OF TREATMENT

In another aspect, provided herein are methods for treating cancer in a subject comprising administering to the subject a therapeutically effective amount of an EZH2 inhibitor in combination with an immune checkpoint therapy.

EZH2 inhibition enhances cytotoxic function of effector T cells and alters the phenotype and function of suppressive regulatory T cells into effector like T cells. Together, EZH2 inhibition enhances T cell mediated anti-tumor immunity that can impact various types of solid tumor, such as prostate and bladder cancer. Tumors for which the present treatment methods are useful include any malignant cell type, such as those found in a solid tumor or a hematological tumor. The cancer may be a cancer comprising suppressive T cells, such as regulatory T cells.

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments, the therapeutically effective amount of the EZH2 inhibitor and/or immune checkpoint therapy is administered to the subject orally, intravenously, intratumorally, or intraperitoneally. Specifically, the EZH2 inhibitor (e.g., CPI-1205) may be administered orally and the immune checkpoint therapy may be administered intravenously. In specific embodiments, an effective dose of CPI-1205 may be an oral twice daily dose of 200 mg, 400 mg 800 mg or 1600 mg. In specific embodiments, the effective dose of CPI-1205 may be provided in 7, 14, 21 or 28 day cycles, or 1, 2, 3, 4, 5, 6, 7, 8, 9, 10, 11 or 12 month cycles. The appropriate dosage of the cell therapy may also be determined based on the type of cancer to be treated, the severity and course of the disease, the clinical condition of the individual, the individual's clinical history and response to the treatment, and the discretion of the attending physician.

In certain embodiments, the EZH2 inhibitor is CPI-1205, PZ-6438, EPZ005687, EPZ011989, Ell, GSK126, GSK343, or UNC 1999. In particular embodiments, the EZH2 inhibitor is CPI-1205 (N-[(4-methoxy-6-methyl-2-oxo-1H-pyridin-3-yl)methyl]-2-methyl-1-[(1R)-1-[1-(2,2,2-trifluoroethyl)piperidin-4-yl]ethyl]indole-3-carboxamide).

In certain embodiments, the immune checkpoint inhibitor inhibits an immune checkpoint protein selected from the group consisting of programmed cell death pathway 1 (PD-1/CD279) and its ligands (PD-L1/CD274 and PD-L2/CD273), cytotoxic T lymphocyte-associated antigen 4 (CTLA-4/CD152), lymphocyte-activation gene 3 (LAG-3/CD223), B and T lymphocyte attenuator (BTLA), T cell immunoreceptor with Ig and immunoreceptor tyrosine-based inhibitory motif (ITIM) domains (TIGIT), T cell immunoglobulin domain and mucin domain 3 (TIM-3/HAVcr2), killer immunoglobulin-like receptor (KIR/CD158), V-domain immunoglobulin suppressor of T cell activation (VISTA), and the adenosine A2a receptor (A2aR).

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In certain embodiments, the immune checkpoint inhibitor is a PD-1 binding antagonist. In certain embodiments, the PD-1 binding antagonist is an anti-PD-1 antibody. In certain embodiments, the anti-PD-1 antibody is selected from the group consisting of nivolumab, pembrolizumab, and CT-011. In certain embodiments, the PD-1 binding antagonist is an immunoadhesin (e.g., an immunoadhesin comprising an extracellular or PD-1 binding portion of PDL1 or PDL2 fused to an immunoglobulin constant region (e.g., an Fc region of an immunoglobulin sequence).

In certain embodiments, the immune checkpoint inhibitor is a CTLA-4 binding antagonist. In certain embodiments, the CTLA-4 binding antagonist is an anti-CTLA-4 antibody. In certain embodiments, the anti-CTLA-4 antibody is selected from the group consisting of ipilimumab and tremelimumab.

A. Combination Therapies

In certain embodiments, the methods provided herein further comprise a step of administering at least one additional therapeutic agent to the subject. All additional therapeutic agents disclosed herein will be administered to a subject according to good clinical practice for each specific composition or therapy, taking into account any potential toxicity, likely side effects, and any other relevant factors.

In certain embodiments, the additional therapy may be immunotherapy, radiation therapy, surgery (e.g., surgical resection of a tumor), chemotherapy, bone marrow transplantation, or a combination of the foregoing. The additional therapy may be targeted therapy. In certain embodiments, the additional therapy is administered before the primary treatment (i.e., as adjuvant therapy). In certain embodiments, the additional therapy is administered after the primary treatment (i.e., as neoadjuvant therapy.

In certain embodiments, the additional therapeutic agent comprises treatment with radiotherapy. In certain embodiments, the radiotherapy is selected from the group consisting of gamma rays (γ-rays), X-rays, microwaves, proton beam irradiation, ultraviolet irradiation, and the directed delivery of radioisotopes to the tumor. In certain embodiments, the radiotherapy comprises treatment with X-rays. In certain embodiments, the X-rays are administered in daily doses of 50 to 200 roentgens over a period of three to four weeks. In certain embodiments, the X-rays are administered in a single dose of 2000 to 6000 roentgens. In certain embodiments, the radiotherapy comprises directed delivery of radioisotopes to the tumor. Dosage ranges for radioisotopes vary widely depending on the half-life of the isotope, the strength and type of radiation emitted, and the degree of uptake by tumor cells, but determination of an appropriate therapeutically effective dose is within the level of ordinary skill in the art.

In certain embodiments, the additional therapeutic agent comprises administration of agents for the treatment of side-effects associated with the primary treatment (e.g., nausea, cachexia, and the like). In certain embodiments, the additional therapy comprises an immunotherapy. In certain embodiments, the additional therapy comprises radiation therapy. In some embodiments, the radiotherapy comprises gamma irradiation. In certain embodiments, the additional therapy comprises surgery. In certain embodiments, the additional therapy comprises a combination of radiation therapy and surgery. In certain embodiments, the additional therapy comprises treatment with a class of chemotherapeutic agent selected from the group consisting of alkylating agents, anthracyclines, cytoskeletal disruptors, epothilones, histone deacetylase inhibitors, topoisomerase I inhibitors, topoisomerase II inhibitors, kinase inhibitors, nucleotide analogs and nucleotide precursor analogs, peptide antibiotics, platinum-based compounds, retinoids, vinca alkaloids and derivatives thereof.

The additional therapies contemplated herein may be administered before, after, or concurrently with administration of the compositions provided herein. In certain embodiments, the additional therapy is administered before the compositions provided herein. In certain embodiments, the additional therapy is administered after the compositions provided herein. In certain embodiments, the additional therapy is administered at one or more intervals before or after administration of the compositions provided herein. Determination of an appropriate interval for administration of an additional therapy such that the subject being treated benefits from the combination therapy is within the level of ordinary skill in the art.

B. Pharmaceutical Compositions

In another aspect, provided herein are pharmaceutical compositions and formulations comprising EZH2 inhibitors and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions, such as normal saline (e.g., 0.9%) and human serum albumin (e.g., 10%). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zinc-protein complexes); and/or non-ionic surfactants such as polyethylene glycol (PEG).

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Combination of EZH2 Inhibitor and Immune Checkpoint Therapy

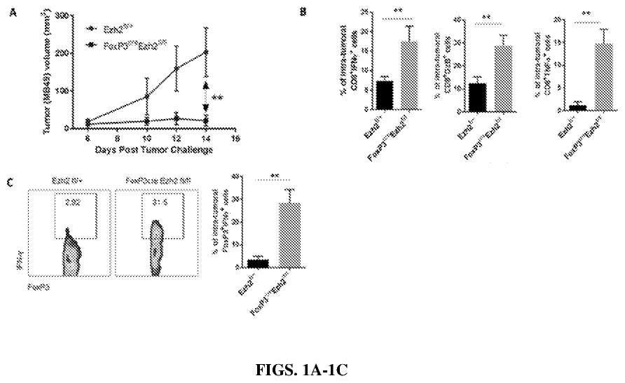

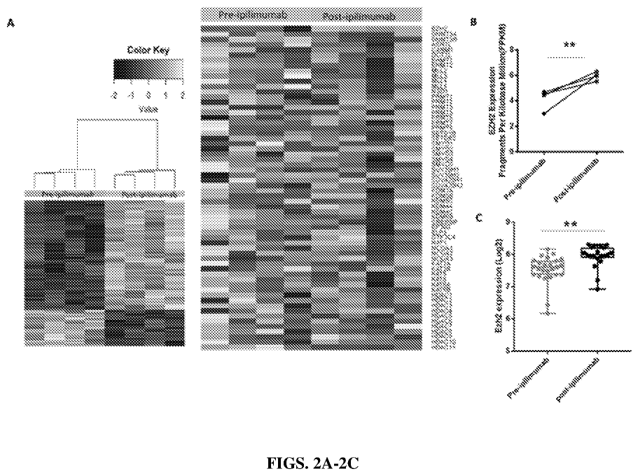

To interrogate the role of EZH2 in T cell mediated anti-tumor immunity, tumor-bearing FoxP3^{cre}EZH2^{fl/fl} C57BL/6 mice were used, which lack EZH2 specifically in the regulatory T cells. FoxP3^{cre}EZH2^{fl/fl} mice and wild type (WT) control C57BL/6 mice were inoculated with a murine bladder cancer cell line (MB49). Tumor growth and immune profiles were studied. FoxP3^{cre}EZH2^{fl/fl} mice had significantly less tumor growth compared with the control mice (FIG. 1A). Analysis of the tumor microenvironment showed a pro-inflammatory state evidenced by increased CD8⁺ interferon (IFN)-γ⁺ cells, CD8⁺ granzyme B (GzB)⁺ cells, and CD8⁺tumor necrosis factor (TNF)-α⁺ cells in tumor-bearing FoxP3^{cre}EZH2^{fl/fl} mice compared with the control mice (FIG. 1B). Increased frequency of cytokine producing CD8 T cells in the FoxP3^{cre} EZH2^{fl/fl} mice suggested EZH2 inhibition in regulatory T cells also had an indirect positive impact on CD8 T cells. Importantly, increased intra-tumoral abundance of FoxP3⁺IFNγ⁺ double positive cells was observed, which implied that the loss of EZH2 function in regulatory T cells can reprogram their phenotype to effector-like T cells and induce robust anti-tumor immunity (FIG. 1C).

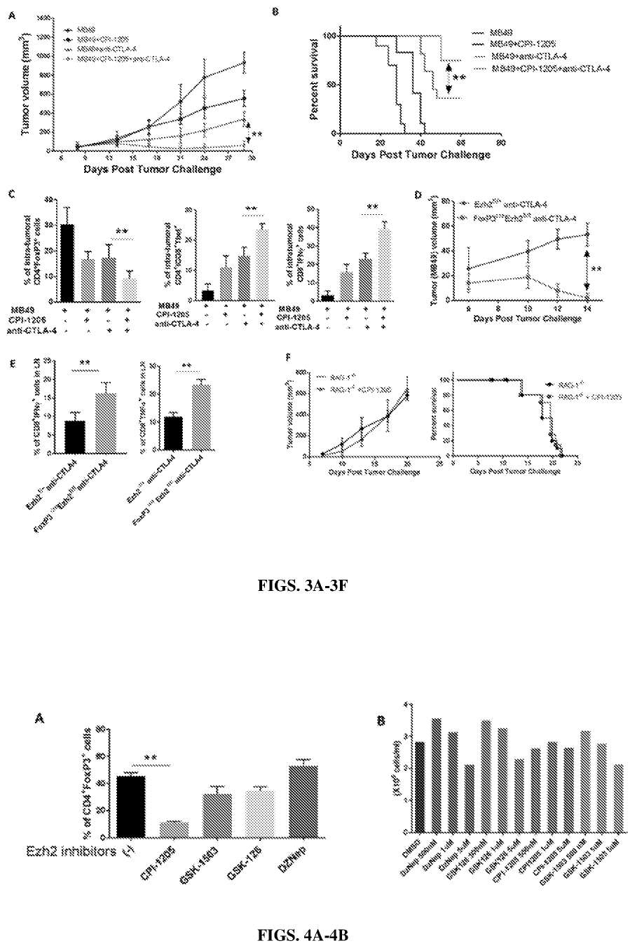

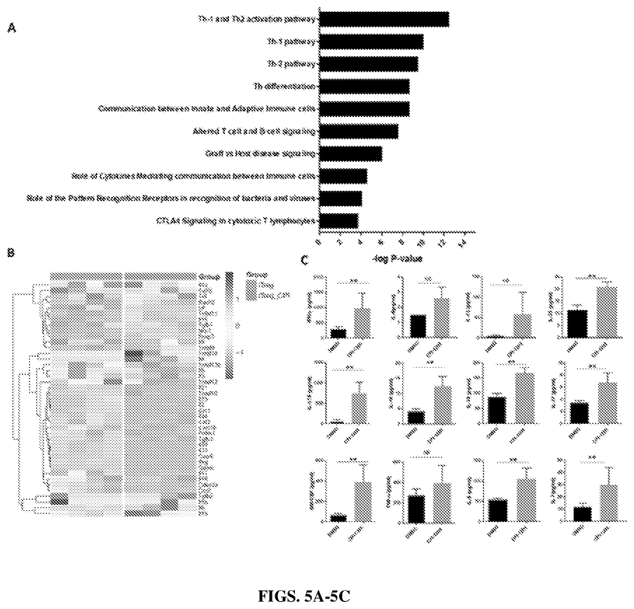

To investigate whether the data observed in the FoxP3^{cre}EZH2^{fl/fl} mouse model could be recapitulated by pharmacological inhibition of EZH2 in T cells, the effect of different EZH2 inhibitors on regulatory T cell differentiation was compared. CPI-1205 was compared with commercially available EZH2 inhibitors. It was found that CPI-1205 had the most striking effect on suppression of murine inducible regulatory T cell (iTreg) differentiation compared with the other EZH2 inhibitors, and none of the inhibitors had a significant effect on regulatory T cell proliferation (FIGS. 4A and B).

To understand CPI-1205-mediated molecular changes underlying the loss of suppressive activity of regulatory T cells, naïve murine T cells (CD4⁺CD25^{low} CD44^{low} CD62^{hi}) from FoxP3-enhanced green fluorescent protein (eGFP) C57BL/6 mice were differentiated into iTregs in the presence or absence of CPI-1205. Purified GFP⁺FoxP3⁺ regulatory T cells were subsequently used for RNA sequencing analysis. Ingenuity Pathway Analysis showed upregulation of pro-inflammatory pathways (Th-1/Th-2) in iTregs following CPI-1205 treatment (FIG. 5A). Examination of the differentially expressed genes demonstrated an increase in effector cytokines and chemokines, while regulatory T cell-specific factors such as FoxP3, BACH2, and neuropilin 1 (NRP1) were decreased following CPI-1205 treatment (FIG. 5B). The cytokine levels were subsequently examined in the culture supernatant of the iTregs that were differentiated with and without CPI-1205. Pro-inflammatory cytokines identified in the RNA sequencing analysis were significantly increased in the supernatant, confirming the gene signature of RNA sequencing data (FIG. 5C). Collectively, these data showed that CPI-1205 treatment influences the differentiation of iTregs and attenuates their suppressive activity. CPI-1205 can also skew the phenotype of iTregs to pro-inflammatory cytokine producing effector-like T cells. In the study, it was noted that EZH2 inhibition leads to decreased expression of not only FoxP3 but also other critical genes, such as NRP1 and BACH2, which are required for regulatory T cell stability (16-18). CPI-1205 treated regulatory T cells seem to acquire the phenotype of so called “exFoxP3” cells which was previously shown to have an activated memory phenotype (Zhou et al., 2009).

T cell activation by CD28 signaling induces EZH2 expression (DuPage et al., 2015). It was postulated that exaggerated CD28 signaling due to anti-CTLA-4 therapy would increase EZH2 expression in T cells. To test this hypothesis, peripheral CD4 T cells were isolated from patients with metastatic melanoma who received ipilimumab as the first line of therapy for metastatic disease, at baseline and after three doses of ipilimumab therapy. RNA sequencing analysis showed differential expression of histone-modifying enzymes (FIG. 2A). Matched paired analysis of histone-modifying enzymes revealed an increase in EZH2 expression in peripheral CD4 T cells following ipilimumab therapy compared with baseline levels (FIG. 2B).

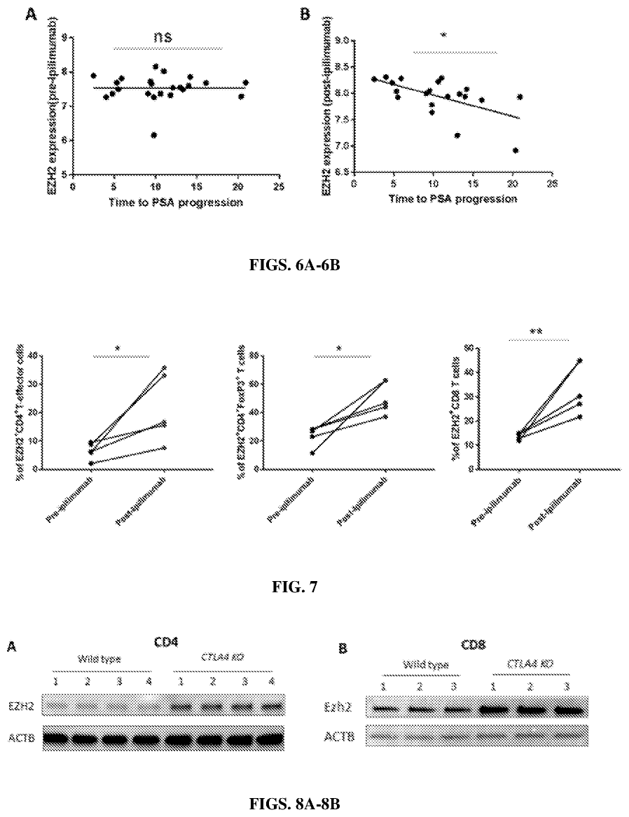

Next, EZH2 expression was confirmed in peripheral CD4 T cells isolated from patients with metastatic prostate cancer at baseline and after three doses of ipilimumab (Subudhi et al., 2016). Like in patients with metastatic melanoma, increased EZH2 expression was found in CD4 T cells following ipilimumab therapy (FIG. 2C) that inversely correlated with prostate-specific antigen progression (FIG. 6). Next, EZH2 expression was evaluated on human CD4 effector T cells (CD4⁺CD45RO⁻CD45RA⁺CCR7⁻), regulatory T cells (CD4⁺CD25⁺FoxP3⁺) and CD8 T cells sorted from peripheral blood of at baseline and post-ipilimumab therapy. It was noted that EZH2 expression increased in all three T cell subtypes following ipilimumab treatment (FIG. 7).

To further elucidate the direct role of CTLA-4 signaling in EZH2 expression in T cells, EZH2 levels were measured in T cells derived from CTLA-4^{-/-} mice compared with wild-type littermate controls. Increased EZH2 levels were observed in both CD4 and CD8 T cells in CTLA-4^{-/-} mice compared with controls (FIG. 8). Altogether, it was noted that blockade of CTLA-4 signaling enhanced EZH2 expression in human and murine T cells. Because of the known effect of CTLA-4 on T cell priming and CD28 signaling, the role of EZH2-mediated T cell function in anti-CTLA-4 therapy was studied.

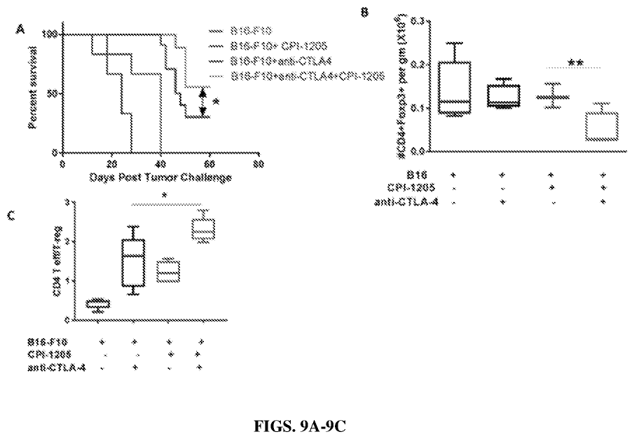

It was evaluated whether the combination of anti-CTLA-4 with CPI-1205 increases the effectiveness of anti-CTLA-4. MB49 (bladder) and B 16-F10 (melanoma) tumor-bearing C57BL/6 mice were treated with anti-CTLA-4 and the EZH2 inhibitor CPI-1205 as monotherapies and in combination. It was found that the combination therapy significantly reduced tumor growth and increased survival in both the MB49 and B 16-F10 models compared with anti-CTLA-4 monotherapy (FIG. 3A, B and FIG. 9A). On interrogating the tumor immune environment, it was noted that combination therapy with CPI-1205 plus anti-CTLA-4 as compared to monotherapy with CPI-1205 or anti-CTLA-4, strikingly reduced the percentage of CD4⁺FoxP3⁺ regulatory T cells and increased the percentage of intra-tumoral effector CD4⁺ICOS⁺T-bet⁺ and CD8⁺IFNγ⁺ cells in both the murine models (FIG. 3C and FIG. 9B), which resulted in an increased ratio of effector T cells to regulatory T cells (FIG. 9C). Furthermore, MB49 tumor-bearing FoxP3^{cre}EZH2^{fl/fl} mice which received anti-CTLA-4 had complete tumor rejection (FIG. 3D) and increased percentages of CD8⁺IFNγ⁺ and CD8⁺TNFα+ cells (FIG. 3E) compared with WT controls.

To determine whether the anti-tumoral effect of combination therapy is exclusively due to effector immune response or is an additive effect of CPI-1205 on tumor cells, Rag 1^{-/-} C57BL/6 mice were used, which have no mature lymphocyte population. MB49 tumor-bearing Ragl^{-/-} mice were treated with CPI-1205. No significant difference was observed in tumor growth and survival in CPI-1205 treated Rag 1^{-/-}mice compared with the untreated group (FIG. 3G), suggesting that in the MB49 bladder cancer model, EZH2 has no effect on the tumor growth.

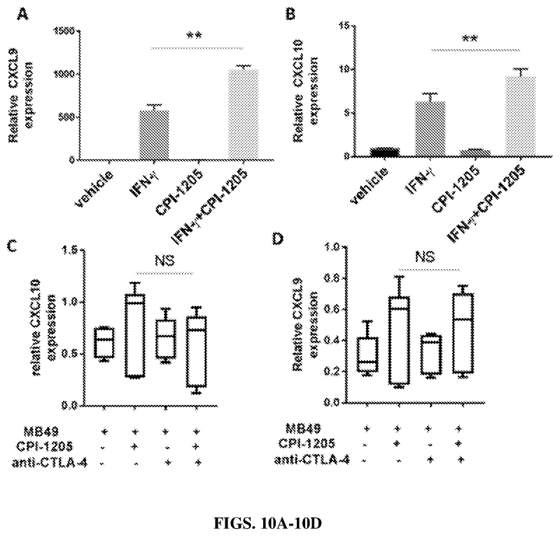

Because EZH2 regulates expression of Th-1 chemokines such as CXCL9 and CXCL10 in the tumor cells (Peng et al., 2015), in vitro (cell line) and in vivo (murine tumor model) experiments were performed to assess the contribution of CXCL9 and CXCL10 in the system. An increase in CXCL9 (in B 16-F10 cells) and CXCL10 (in MB-49 cells) was observed following stimulation with IFN-γ plus CPI-1205 in vitro (FIGS. 10A and B). Additionally, while CPI-1205 treatment increased the expression of CXCL9 and CXL10 expression in MB49 tumor bearing mice, combination treatment with the anti-CTLA4 antibody plus CPI-1205 did not further increase tumor CXCL9 or CXCL10 as compared to the single agent CPI-1205 treatment group (FIGS. 10C and D). Overall, these observations suggest that although CPI-1205 treatment might enhance chemokine-mediated T cell infiltration of tumors, the latter mechanism does not seem to significantly contribute to the enhanced anti-tumor immunity observed with the combination therapy. The contribution of other immune cell subsets such as B cells and myeloid cells was also measured in CPI-1205 mediated anti-tumor immunity. Although no difference in B cell abundance was noted, CPI-1205 treatment effected certain subsets of myeloid cells which warrant further investigations.

In conclusion, the direct role of EZH2-mediated reprogramming of T cells in anti-tumor immunity was demonstrated, especially in the context of anti-CTLA-4 therapy. Ipilimumab leads to a compensatory increase in EZH2 expression in T cells, whereas inhibition of EZH2 improves response to anti-CTLA-4 through modulation of tumor cytotoxic effector T cells and altering the phenotype of regulatory T cells into effector-like T cells. No immune-related toxicities were observed in mice treated anti-CTLA-4 plus CPI-1205. Ipilimumab has been safely given to patients in combination with other immune checkpoint therapy such as Nivolumab with manageable side effect profile. Importantly, CPI-1205 is well tolerated when given on continuous daily dosing in patients with no dose-limiting toxicities. Thus, the mechanistic insight gathered in this study provides a strong rationale to initiate a clinical trial with CPI-1205 plus ipilimumab in patients with primary or adaptive resistance to anti-CTLA-4 therapy.

### Example 2—Materials and Methods

Patient samples. Metastatic melanoma samples were obtained from patients who received ipilimumab as the standard of care. Prostate cancer samples were obtained from a phase II study of androgen deprivation therapy in combination with ipilimumab from metastatic non-castrate disease in patients who received androgen deprivation therapy within 1 month of starting ipilimumab (#NCT01377389). Samples were collected at baseline (before patients started ipilimumab) and after three of ipilimumab.

Mice. C57BL/6 (5-7 weeks) mice were purchased from the National Cancer Institute (Frederick, Md.). Foxp3^{cre}, EZH2^{fl/fl}, Foxp3-eGFP, and Ragl^{-/-} mice, all of the C57BL/6 background, were obtained from The Jackson Laboratory (Bar Harbor, Me.). FoxP3^{cre}EZH2^{fl/fl} mice were generated by breeding Foxp3^{cre} and EZH2^{fl/fl} C57BL/6 mice. All mice were kept in specific pathogen-free conditions in the Animal Resource Center at The University of Texas MD Anderson Cancer Center. Animal protocols were approved by the Institutional Animal Care and Use Committee of The University of Texas MD Anderson Cancer Center.

Cell lines and tumor model. Murine bladder cancer cell line (MB49) were provided by Dr. A. Kamat (at The University of Texas MD Anderson Cancer Center) and murine melanoma cell line B16-F10 was obtained from Dr. I. Fidler (The University of Texas MD Anderson Cancer Center). 2×10⁵ (MB49) or 2×10⁵ (B16-F10) cells were injected subcutaneously or intradermally respectively, in the flanks of C57BL/6 mice (5 or 10 mice per group). EZH2 inhibitor (CPI-1205; Constellation Pharmaceuticals Cambridge, Mass.) (200 mg/kg) was administered twice daily through oral gavage from day 3 to the end of the experiment. On day 7 after tumor inoculation, when tumors became palpable, mice were injected intraperitoneally with α-CTLA-4 (clone 9H10, Bio X Cell, NH) (100 μg/mouse). A second dose of anti-CTLA-4 was administered on day 9.

Tissue processing and flow cytometry. Tumor bearing mice were sacrificed on day 14 and single cell suspensions from spleen, lymph node and tumor were prepared as described previously (1). Single cell suspension from frozen human peripheral blood samples were performed as described previously (2). For flow cytometry based analysis of surface markers, cells were stained in phosphate-buffered saline containing 5% bovine serum albumin with LIVE/DEAD yellow dye (Thermo Fisher Scientific), anti-CD45 (Biolegend, 30-F11), anti-CD4 (BioLegend, RM4-5), anti-CD8a (BioLegend, 53-6.7), anti-CD44 (eBioscience, 1M7), anti-CD62L (BioLegend, MEL-14), anti-CD25 (BioLegend, 3C7), anti-ICOS (eBioscience, 7E.17G9), anti-CD27 (eBioscience, LG.7F9), anti-CD39 (eBioscience, 24DMS1), anti-FR4 (eBioscience, eBiol2A5), CD4 (BioLegend, OKT4), CD25 (BioLegend, M-A251), FoxP3 (eBioscience, 236A/E7), anti-CD3 (BioLegend, UCHT1), anti-CD8a (BioLegend, RPA-T8), anti-CD25 (BioLegend, BC96), anti-CD197 (BioLegend, CCR7), anti-CD45RA (BioLegend, HI100), and anti-CD45RO (BioLegend, UCHL1) on ice for 30 minutes. Staining with intracellular anti-Foxp3 (eBioscience, FJK-16 s), antiIL-2 (eBioscience, PC61.5), antiKi-67 (eBioscience, SolA15), antiIFN-γ (BioLegend, XMG1.2), anti-TNF-α (BioLegend, MP6-XT22), and anti-EZH2 (Cell Signaling Technology, D2C9) were analyzed by flow cytometry according to the manufacturers’ instructions. Flow cytometry data were acquired on BD LSR II (BD Biosciences) and analyzed using FlowJo software (Tree Star, Ashland, Oreg.).

In vitro T cell assays. Human peripheral regulatory T cells were isolated from peripheral blood mononuclear cells by enriching for CD4⁺CD127^{low} CD25⁺ cells using the EasySep Human Regulatory T Cell Isolation Kit (STEMCELL Technologies, Cambridge, Mass.). For mouse in vitro regulatory T cell differentiation, CD4⁺CD25^{low} CD44^{low} CD62L^{hi}naïve splenic T cells were isolated from C57BL/6 mice using BD FACSAria (BD Biosciences, San Jose, Calif.). The cells were then differentiated in the presence of recombinant murine IL-2 (R&D, 402-ML), tumor growth factor-β (TGF-β, R&D 7666-MB), CPI-1205 or dimethyl sulfoxide (DMSO) and cultured for 6 days.

For T cell suppression assays, autologous CD4⁺ conventional T cells (Tconv) were isolated by magnetic separation (STEMCELL Technologies) from cryopreserved peripheral blood mononuclear cells rested overnight. Tconv were labeled with CellTrace Violet (Thermo Fisher Scientific, Waltham, Mass.) as instructed by the manufacturer. Tconv and autologous induced regulatory T cells were co-cultured in various ratios in ImmunoCult medium (STEMCELL Technologies). Suppression was subsequently analyzed by flow cytometry (CellTrace Violet based proliferation in combination with fixable live/dead dye and CD4, CD25, and FOXP3 staining).

T cell cytotoxicity assay was carried out using activated effector T cells (isolated with EasySep Human Naïve CD4⁺ T Cell Isolation Kit and EasySep Human CD8⁺ T Cell Enrichment Kit; STEMCELL Technologies). Purified naïve CD4 and total CD8 T cells were pre-activated with plate-bound anti-CD3/soluble anti-CD28 for 6 days in the presence or absence of CPI-1205. Activated T cells were then washed and challenged with Nalm-6 target tumor cells, then labeled with CellTrace violet to enable their subsequent discrimination from effector cells. The T cell:tumor cell co-cultures were conducted at different effector:target (E:T) ratios, in the presence of the blinatumomab antibody (10 ng/ml) and CPI-1205 for 20 hours as previously described (3). Target cell death was determined by summing the percentage of apoptotic (Annexin V⁺) and dead or dying (7AAD+) target (CellTrace violet⁺) cells after overnight culture.

RNA sequencing. RNA was extracted from peripheral CD4 T cells isolated from patients with metastatic melanoma and from in vitro differentiated GFP⁺ regulatory T cells. RNA sequencing reactions were performed by Active Motif (Carlsbad, Calif.) and Ocean Ridge Biosciences (Deerfield, Fla.) using an Illumina HiSeq 50-bp platform. For bioinformatics analysis, the raw reads were aligned to the mm10 reference genome using TopHat2. HTSeq count was used to count the raw reads that were uniquely mapped to each gene. DESeq2 was then applied to normalize the raw read counts and identify the differentially expressed genes between the groups. The beta-uniform mixture model was used to fit the p value distribution for multiple testing adjustments. The lists of differentially expressed genes were then used as input into Ingenuity Pathway Analysis for pathway analysis. RNA-sequencing data were deposited in the NCBI's Gene Expression Omnibus database.

Quantitative Real-time RTPCR. RNA is extracted using RNeasy Plus Micro Kit (QIAGEN) and reverse-transcription reactions were performed using miScript II RT Kit (QIAGEN), RT-PCR was performed using 7500 Fast Real-time PCR System (Applied Biosystems). The specific primers (origene) used for the assays are listed below.

Primer sequences:
Actin
(F: CATTGCTGACAGGATGCAGAAGG, SEQ ID NO: 1
R: TGCTGGAAGGTGGACAGTGGAG) SEQ ID NO: 2
CXCL9
(F: CCTAGTGATAAGGAATGCACGATG (SEQ ID NO: 3,
R: CTAGGCAGGTTTGATCTCCGTTC) SEQ ID NO: 4
CXCL10
(F: ATCATCCCTGCGAGCCTATCCT (SEQ ID NO: 5,
R: GACCTTTTTTGGCTAAACGCTTTC) SEQ ID NO: 6

Western blot. Cell lysates were prepared from mouse CD4 and CD8 T cells, Western blot was performed using a standard Western blot protocol with EZH2 (Cell Signaling Technology, D2C9). Quantitation was performed using LI-COR Biosciences (Lincoln, Nebr.) Image Studio software.

Statistical analysis. All data are representative of at least two to three independent experiments with 5-10 mice in each in vivo experiment. The data are expressed as mean+standard error of the mean (SEM) and were analyzed using Prism 5.0 statistical analysis software (GraphPad Software, La Jolla, Calif.). Student t-tests (two tailed), ANOVA, and Bonferroni multiple comparison tests were used to identify significant differences (p<0.05) between treatment groups. Ingenuity Pathway Analysis was done on data obtained from RNA sequencing using Ingenuity Pathway Analysis software (QIAGEN, Hilden, Germany).

Statistical analysis: All data are shown as the mean+SEM, a 2-tailed Student's t test or 1-way ANOVA or Bonferroni multiple comparison tests to identify significant differences (p<0.05) between treatment groups. Ingenuity Pathway Analysis was done on data obtained from RNA sequencing using Ingenuity Pathway Analysis software (QIAGEN, Hilden, Germany). A p value of less than 0.05 was considered statistically significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arvey et al., Nature immunology. 2014; 15(6):580-7.
Cao et al., Science. 2002; 298(5595):1039-43.
Coit et al., Arthritis Rheumatol. 2016; 68(9):2200-9.
DuPage et al., Immunity. 2015; 42(2):227-38.
Kanno et al., Annual review of immunology. 2012; 30(707-31.
Kwon et al., Nature immunology. 2017; 18(11):1238-48.
Margueron and Reinberg. Nature. 2011; 469(7330):343-9.
Nagarsheth et al., Cancer research. 2015.
Peng et al., Nature. 2015; 527(7577):249-53.
Remington's Pharmaceutical Sciences 22^{nd} edition, 2012
Sarmento et al., The Journal of biological chemistry. 2017; 292(2):706-22.
Subudhi et al., Proceedings of the National Academy of Sciences of the United States of America. 2016; 113(42):11919-24.
Tumes et al., Immunity. 2013; 39(5):819-32.
Wei et al., Immunity. 2009; 30(1):155-67.
Yang et al., Scientific reports. 2015; 5:10643.
Zhang et al., Blood. 2014; 124(5):737-49.
Zhou et al., Nature immunology. 2009; 10(9):1000-7.
Zingg et al., Cell reports. 2017; 20(4):854-67.

What is claimed is:

1. A method of treating cancer in a subject comprising administering an effective amount of CPI-1205 in combination with an immune checkpoint inhibitor to the subject.

2. The method of claim 1, wherein the subject has one or more tumors.

3. The method of claim 2, wherein the administration of CPI-1205 and immune checkpoint inhibitor results in reduced tumor growth or a reduction in tumor mass.

4. The method of claim 3, wherein the administration of CPI-1205 and immune checkpoint inhibitor results in greater reduction in tumor growth or greater reduction in tumor mass relative to administration of immune checkpoint inhibitor therapy alone.

5. The method of claim 1, wherein the subject is resistant to an immune checkpoint inhibitor.

6. The method of claim 1, wherein the subject is human.

7. The method of claim 1, wherein more than one immune checkpoint inhibitor is administered.

8. The method of claim 1, wherein the CPI-1205 and/or the immune checkpoint inhibitor are administered orally, intravenously, intraperitoneally, intratracheally, intratumorally, intramuscularly, endoscopically, intralesionally, percutaneously, subcutaneously, regionally, or by direct injection or perfusion.

9. The method of claim 1, wherein the CPI-1205 is administered orally and the immune checkpoint inhibitor is administered intravenously.

10. The method of claim 1, wherein the CPI-1205 and/or the immune checkpoint inhibitor are administered more than once.

11. The method of claim 1, wherein the CPI-1205 and/or the immune checkpoint inhibitor are administered daily.

12. The method of claim 1, wherein the CPI-1205 and the immune checkpoint inhibitor are administered concurrently.

13. The method of claim 1, wherein the CPI-1205 is administered before the immune checkpoint inhibitor.

14. The method of claim 1, wherein the CPI-1205 is administered after the immune checkpoint inhibitor.

15. The method of claim 1, wherein the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR).

16. The method of claim 1, wherein the immune checkpoint inhibitor is a PD-1 inhibitor.

17. The method of claim 16, wherein the PD-1 inhibitor is nivolumab, pembrolizumab, CT-011, BMS 936559, MPDL328OA or AMP-224.

18. The method of claim 1, wherein the immune checkpoint inhibitor is a CTLA-4 inhibitor.

19. The method of claim 18, wherein the CTLA-4 inhibitor is ipilimumab or tremelimumab.

20. The method of claim 19, wherein the CTLA-4 inhibitor is ipilimumab.

21. The method of claim 20, wherein CPI-1205 is administered at an 800 mg twice daily oral dose and ipilimumab is administered intravenously at a dose of 3 mg/kg every 3 weeks.

22. The method of claim 1, wherein the administration results in an increase in CD8⁺ interferon (IFN)-γ⁺ T cells, CD8⁺ granzyme B (GzB)⁺ T cells and/or CD8⁺ tumor necrosis factor (TNF)-α⁺ T cells.

23. The method of claim 2, wherein the administration results in an increase of intra-tumoral T cells.

24. The method of claim 23, wherein the intra-tumoral T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺.

25. The method of claim 2, wherein the administration results in an increased ratio of effector T cells to regulatory T cells intratumorally.

26. The method of claim 2, wherein the administration results in increased infiltration of T cells into the one or more tumors.

27. The method of claim 26, wherein the T cells are effector T cells.

28. The method of claim 26, wherein the T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺.

29. The method of claim 1, wherein the administration results in a decrease in suppressive T cells.

30. The method of claim 1, wherein the administration results in a decrease in inducible regulatory T cell differentiation.

31. The method of claim 1, wherein the administration results in a decrease in FOXP3, NRP1, and/or BACH2 expression.

32. The method of claim 31, wherein the decrease in FOXP3, NRP1, and/or BACH2 expression is in T cells.

33. The method of claim 1, wherein the cancer is bladder cancer, melanoma or prostate cancer.

34. The method of claim 1, further comprising the step of administering at least one additional therapeutic agent to the subject.

35. The method of claim 1, wherein the subject receives at least one additional type of therapy.

36. The method of claim 35, wherein the at least one additional type of therapy is selected from the group consisting of chemotherapy, radiotherapy, and immunotherapy.

37. A method of treating cancer in a subject comprising administering an EZH2 inhibitor to the subject, wherein the patient has been determined to be resistant to immune checkpoint therapy.

38. The method of claim 37, wherein the EZH2 inhibitor is CPI-1205.

39. The method of claim 37, further comprising administering an immune checkpoint inhibitor.

40. The method of claim 39, wherein the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR).

41. The method of claim 39, wherein the immune checkpoint inhibitor is a PD-1 inhibitor.

42. The method of claim 41, wherein the PD-1 inhibitor is nivolumab, pembrolizumab, CT-011, BMS 936559, MPDL328OA or AMP-224.

43. The method of claim 39, wherein the immune checkpoint inhibitor is a CTLA-4 inhibitor.

44. The method of claim 43, wherein the CTLA-4 inhibitor is ipilimumab or tremelimumab.

45. The method of claim 44, wherein the CTLA-4 inhibitor is ipilimumab.

46. The method of claim 37, wherein the subject is human.

47. The method of claim 37, wherein the cancer is bladder cancer, melanoma or prostate cancer.

48. A method of depleting regulatory T cells (Tregs) in a subject comprising administering an EZH2 inhibitor to the subject.

49. The method of claim 48, wherein the subject is human.

50. The method of claim 48, wherein the cancer is bladder cancer, melanoma or prostate cancer.

51. The method of claim 48, wherein the Tregs are inducible regulatory T cells.

52. The method of claim 48, wherein the Tregs are FoxP3⁺ Tregs.

53. The method of claim 48, wherein the EZH2 inhibitor is CPI-1205.

54. The method of claim 48, further comprising administering an immune checkpoint inhibitor.

55. The method of claim 54, wherein the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR).

56. The method of claim 54, wherein the immune checkpoint inhibitor is a PD-1 inhibitor.

57. The method of claim 56, wherein the PD-1 inhibitor is nivolumab, pembrolizumab, CT-011, BMS 936559, MPDL328OA or AMP-224.

58. The method of claim 54, wherein the immune checkpoint inhibitor is a CTLA-4 inhibitor.

59. The method of claim 58, wherein the CTLA-4 inhibitor is ipilimumab or tremelimumab.

60. The method of claim 58, wherein the CTLA-4 inhibitor is ipilimumab.

61. A pharmaceutical composition comprising CPI-1205 and an immune checkpoint inhibitor.

62. The pharmaceutical composition of claim 61 for use in the treatment of cancer.

63. The use of a therapeutically effective amount of the CPI-1205 and an immune checkpoint inhibitor for the treatment of cancer.

64. The use of claim 63, wherein the cancer is bladder cancer, melanoma or prostate cancer.

65. A composition comprising a therapeutically effective amount of CPI-1205 and an immune checkpoint inhibitor for the treatment of cancer in a subject.

66. Use of an Enhancer of zeste homolog 2 (EZH2) inhibitor and an immune checkpoint inhibitor in the manufacture of a medicament for the treatment of cancer.

67. The use of claim 66, wherein the EZH2 inhibitor is CPI-1205.

68. The use of claim 67, wherein the immune checkpoint inhibitor is ipilimumab.

69. The use of claim 66, wherein the cancer is bladder cancer, melanoma or prostate cancer.

70. A method of treating cancer in a subject comprising inhibiting Enhancer of zeste homolog 2 (EZH2) function in the subject.

71. The method of claim 70, wherein the subject is human.

72. The method of claim 70, wherein the subject has one or more tumors.

73. The method of claim 70, wherein EZH2 function is inhibited in regulatory T cells.

74. The method of claim 73, wherein the regulatory T cells are inducible regulatory T cells.

75. The method of claim 70, wherein EZH2 function is inhibited by administration of an effective amount of an EZH2 inhibitor.

76. The method of claim 75, wherein the EZH2 inhibitor is CPI-1205.

77. The method of any of claims 70-76, wherein the subject is administered an immune checkpoint inhibitor.

78. The method of any of claims 70-77, wherein EZH2 inhibition results in a decrease of one or more regulatory T cell specific factors.

79. The method of any of claims 70-77, wherein EZH2 inhibition results in a decrease in expression in T cells of one or more genes selected from the group consisting of Il1a, Cd70, Tnf, Bach2, Lif, Tnsf11, Il16, Tgfb1, Nrp1, Foxp3, Il9, Tnfsf9 and Tnfsf18.

80. The method of any of claims 70-77, wherein EZH2 inhibition results in an increase in expression in T cells of one or more genes selected from the group consisting of 114, Tnfsf13b, Il5, Il3, Tnfsf12, Il21, Tnfsf10, Il13, Il2, Ccr1, Il24, Csf2, Cxcl10, Prbm1, Tgfb3, Il10, Il33, Cxcr6, Ifng, GzB, Il11, Il18, Cdkn2a, ccr2, Tgfb2, Il1b, Il16 and Il15.

81. The method of claim 78, wherein the one or more regulatory T cell specific factors is FoxP3, BACH2 or neuropilin 1.

82. The method of any of claims 70-77, wherein EZH2 inhibition results in an increase of one or more effector cytokines or chemokines selected from the group consisting of IFN-gamma, IL-25, Il-17A, IL-10, IL-18, IL-27, GM-CSF, IL-9 and IL-7.

83. The method of any of claims 70-82, wherein EZH2 inhibition attenuates inducible regulatory T cell suppressive activity.

84. The method of any of claims 70-83, wherein the cancer is bladder cancer, prostate cancer or melanoma.

85. The method of any of claims 77-84, wherein the subject has one or more tumors and inhibition of EZH2 function and administration of the immune checkpoint inhibitor results in an increase in effector T cells in the tumor.

86. A method of potentiating immune checkpoint inhibitor therapy in a subject comprising administering an effective amount of an Enhancer of zeste homolog 2 (EZH2) inhibitor.

87. The method of claim 86, wherein the subject has been administered, is concurrently being administered or will be administered an immune checkpoint inhibitor.

88. The method of claim 86, wherein the subject has one or more tumors.

89. The method of claim 88, wherein the EZH2 inhibitor is CPI-1205.

90. The method of claim 89, wherein the administration of CPI-1205 and immune checkpoint inhibitor results in reduced tumor growth or a reduction in tumor mass.

91. The method of claim 90, wherein the administration of CPI-1205 and immune checkpoint inhibitor results in greater reduction in tumor growth or greater reduction in tumor mass relative to administration of immune checkpoint inhibitor therapy alone.

92. The method of claim 86, wherein the subject is human.

93. The method of claim 86, wherein more than one immune checkpoint inhibitor is administered.

94. The method of claim 89, wherein the CPI-1205 is administered orally and the immune checkpoint inhibitor is administered intravenously.

95. The method of claim 89, wherein the CPI-1205 and/or the immune checkpoint inhibitor are administered more than once.

96. The method of claim 89, wherein the CPI-1205 and/or the immune checkpoint inhibitor are administered daily.

97. The method of claim 89, wherein the CPI-1205 and the immune checkpoint inhibitor are administered concurrently.

98. The method of claim 89, wherein the CPI-1205 is administered before the immune checkpoint inhibitor.

99. The method of claim 89, wherein the CPI-1205 is administered after the immune checkpoint inhibitor.

100. The method of claim 86, wherein the immune checkpoint inhibitor inhibits an immune checkpoint protein or ligand thereof selected from the group consisting of CTLA-4, PD-1, PD-L1, PD-L2, LAG-3, BTLA, B7H3, B7H4, TIM3, KIR, or adenosine A2a receptor (A2aR).

101. The method of claim 89, wherein the immune checkpoint inhibitor is a PD-1 inhibitor.

102. The method of claim 101, wherein the PD-1 inhibitor is nivolumab, pembrolizumab, CT-011, BMS 936559, MPDL328OA or AMP-224.

103. The method of claim 89, wherein the immune checkpoint inhibitor is a CTLA-4 inhibitor.

104. The method of claim 103, wherein the CTLA-4 inhibitor is ipilimumab or tremelimumab.

105. The method of claim 104, wherein the CTLA-4 inhibitor is ipilimumab.

106. The method of claim 89, wherein the administration of CPI-1205 and immune checkpoint inhibitor results in an increase in CD8⁺ interferon (IFN)-γ⁺ T cells, CD8⁺ granzyme B (GzB)⁺ T cells and/or CD8⁺ tumor necrosis factor (TNF)-α⁺ T cells.

107. The method of claim 89, wherein the administration of CPI-1205 and immune checkpoint inhibitor results in an increase of intra-tumoral T cells.

108. The method of claim 107, wherein the intra-tumoral T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺.

109. The method of claim 89, wherein the administration of CPI-1205 and immune checkpoint inhibitor results in an increased ratio of effector T cells to regulatory T cells intratumorally.

110. The method of claim 89, wherein the administration of CPI-1205 and immune checkpoint inhibitor results in increased infiltration of T cells into the one or more tumors.

111. The method of claim 110, wherein the T cells are effector T cells.

112. The method of claim 110, wherein the T cells are CD8⁺ interferon (IFN)-γ⁺, CD8⁺ granzyme B (GzB)⁺, CD8⁺ tumor necrosis factor (TNF)-α⁺ or FoxP3⁺IFNγ⁺.

113. The method of claim 89, wherein the administration of CPI-1205 and immune checkpoint inhibitor results in a decrease in suppressive T cells.

114. The method of claim 89, wherein the administration of CPI-1205 and immune checkpoint inhibitor results in a decrease in inducible regulatory T cell differentiation.

115. The method of claim 89, wherein the administration of CPI-1205 and immune checkpoint inhibitor results in a reduction in the percentage of CD4⁺FoxP3⁺ regulatory T cells intratumorally.

116. The method of claim 89, wherein the administration of CPI-1205 and immune checkpoint inhibitor results in an increase in the percentage of intra-tumoral CD4⁺ICOS⁺T-bet⁺ effector T cells.

117. The method of claim 89, wherein the administration of CPI-1205 and immune checkpoint inhibitor results in an increase in the percentage of intra-tumoral CD8⁺IFNγ⁺ effector T cells.

118. The method of claim 89, wherein the administration of CPI-1205 and immune checkpoint inhibitor results in an increased percentage of CD8⁺IFNγ⁺ T cells, CD8⁺TNFα⁺ T cells or both, in lymph nodes.

119. A method of treating cancer in a subject comprising administering CPI-1205 to a subject with cancer or being treated for cancer to inhibit an immune checkpoint inhibitor therapy mediated increase in Enhancer of zeste homolog 2 (EZH2) activity.

120. The method of claim 119, wherein the immune checkpoint inhibitor therapy mediated increase in EZH2 activity is in T cells.

121. The method of claim 120, wherein the T cells are regulatory or suppressive T cells.

122. A method of treating cancer in a subject, comprising administering an EZH2 inhibitor to a subject with cancer or being treated for cancer to inhibit an immune checkpoint inhibitor therapy mediated increase in Enhancer of zeste homolog 2 (EZH2) activity.

123. The method of claim 122, wherein the immune checkpoint inhibitor therapy mediated increase in EZH2 activity is in T cells.

124. The method of claim 123, wherein the T cells are regulatory or suppressive T cells.

125. A method of identifying a cancer patient as a candidate for treatment with an Enhancer of zeste homolog 2 (EZH2) inhibitor comprising testing a sample from the patient to determine if the patient has elevated EZH2 activity, wherein if the sample exhibits elevated EZH2 activity the patient is a candidate for EZH2 inhibitor therapy.

126. The method of claim 125, wherein the patient is receiving immune checkpoint inhibitor therapy.

127. The method of claim 125, wherein the patient will receive immune checkpoint inhibitor therapy.

128. The method of claim 126, wherein the elevated EZH2 activity is a result of the immune checkpoint inhibitor therapy.

129. The method of claim 128, wherein the EZH2 inhibitor is CPI-1205.

130. The method of claim 128, wherein the immune checkpoint inhibitor therapy is ipilimumab.

131. The method of claim 126, wherein the elevated EZH2 activity is determined in T cells.

132. The method of claim 126, wherein the sample is a blood sample.

133. The method of claim 126, wherein the sample is a T cell sample.

134. The method of claim 126, wherein the elevated EZH2 activity is determined by measuring a decrease of one or more effector cytokines or chemokines selected from the group consisting of IFN-gamma, IL-25, Il-17A, IL-10, IL-18, IL-27, GM-CSF, IL-9 and IL-7 in the sample.

135. The method of claim 126, wherein the elevated EZH2 activity is determined by measuring an increase in expression in T cells of one or more genes selected from the group consisting of Il1a, Cd70, Tnf, Bach2, Lif, Tnsf1, Il16, Tgfb1, Nrp1, Foxp3, Il9, Tnfsf9 and Tnfsf18.

136. The method of claim 126, wherein the elevated EZH2 activity is determined by measuring a decrease in expression in T cells of one or more genes selected from the group consisting of Il4, Tnfsf13b, Il5, Il3, Tnfsf12, Il21, Tnfsf10, Il13, Il2, Ccr1, Il24, Csf2, Cxcl10, Prbm1, Tgfb3, Il10, Il33, Cxcr6, Ifng, GzB, Il11, Il18, Cdkn2a, ccr2, Tgfb2, Il1b, Il16 and Il15.

137. The method of claim 126, wherein if the patient is determined to be a candidate for EZH2 therapy the patient is administered an oral twice daily dose of 800 mg of CPI-1205.

138. The method of claim 126, wherein the elevated EZH2 activity is determined relative to EZH2 activity in a patient sample collected prior to the patient receiving immune checkpoint inhibitor therapy.
